(19) United States
(12) Patent Application Publication
(10) Pub. No.: US 2026/0036537 A1
(43) Pub. Date: Feb. 5, 2026

(54) SYSTEMS FOR NON-DESTRUCTIVE IN-SITU DETERMINATION OF R-VALUES OF MATERIALS AND METHODS THEREOF

(71) Applicant: National Research Council of Canada, Ottawa (CA)

(72) Inventors: Sudhakar Molleti, Ottawa (CA); Phaik Hoon Yew, Ottawa (CA); Appupillai Baskaran, Ottawa (CA)

(21) Appl. No.: 18/998,887

(22) PCT Filed: Jul. 19, 2023
(86) PCT No.: PCT/CA2023/050973

§ 371 Date: Jan. 27, 2025

Related U.S. Application Data
(60) Provisional application No. 63/392,859, filed on Jul. 27, 2022.

(51) Int. Cl. G01N 25/18; G01N 33/38

(52) U.S. Cl. CPC G01N 25/18; G01N 33/383

(57) ABSTRACT

A method for non-destructive in-situ determination of the R-value of a material at a mean temperature comprises maintaining three or more steady-state temperatures, each for a respective measurement period, measuring temperature and heat flow through the material, calculating R-values at each steady-state temperature and determining the R-value of the material at the mean temperature from the calculated R-values. A system for non-destructive in-situ determination of the R-value of a material at a mean temperature comprises a housing defining a chamber, a temperature control system for heating and cooling the chamber between three or more steady-state temperatures, heat flux transducers, temperature sensors and a computing device from controlling the temperature control system and comprising a data acquisition system for recording measurements from the heat flux transducers and the temperature sensors.

### CROSS-REFERENCE TO RELATED APPLICATION

This application claims priority to and benefit from U.S. patent application Ser. No. 63/392,859 filed on Jul. 27, 2022, which is hereby incorporated by reference in its entirety.

### FIELD OF THE DISCLOSURE

The present disclosure relates generally to measuring thermal parameters of materials, and in particular to measuring R-values of roofing materials.

### BACKGROUND

Existing methods of determining R-values of materials, including materials for roof assemblies, include (1) destructive approaches, e.g. whereby test cuts are made in the roof assembly and samples are tested in a laboratory to determine thermal resistance; (2) endoscopy approaches, whereby based on the components constituting the roof assembly, as per drawings and manufactures' material specifications, the thermal performance is estimated; or (3) heat flux transducer approaches, whereby a heat flux transducer is installed on the exterior surface (membrane) or the interior surface (on the structural substrate) of the roof and a relationship is determined between temperature gradient and heat flux to estimate the thermal performance.

Destructive and endoscopy approaches have associated replacement costs (e.g., cutting, replacing, and inspection of the roof portions), compromise waterproofing functionality of the roof membrane, and introduce uncertainty (e.g., lack of drawings, temperature dependent thermal performance of the materials, and aging of the material) in the estimation of R-value of the roof assembly. Conventional measurements via heat flux transducers installed on the roof's exterior surface (membrane) or on the interior surface (on the structural substrate) can have uncertainty of at least around 15% due to the influence of boundary conditions and, when combined with the daily heat gains and temperature fluctuations (outdoor and indoor), the total accuracy may be significantly impacted.

It is therefore an object to provide improved methods to determine the thermal performance of a material, and in particular a roof assembly's thermal performance.

### SUMMARY

Embodiments disclosed herein relate to non-destructive in-situ methods and systems for determining the thermal performance (e.g. R-value) of a material. In particular embodiments, the methods and systems herein may be used to determine a roof assembly's design-U value or insulation R-values for energy certification, energy audits, and design for roof replacement or roof recovering. Methods and systems disclosed herein include a temperature control system for heating and cooling a chamber used for obtaining measurements.

The present disclosure recognizes that there are problems in the current existing technology in respect of methods of determining R-values of materials, and in particular roofs or roofing materials of existing buildings. Advantageously, the methods and systems disclosed herein are non-destructive and can be performed in-situ, for example on an already constructed and/or occupied building in which the internal temperature is around standard room temperature of between about 20 and about 25 degrees Celsius.

According to one aspect of this disclosure, there is provided a method for non-destructive in-situ determination of the R-value of a material at a mean temperature, the method including the steps of maintaining three or more steady-state temperatures at a first side of the material, wherein each of the three or more steady-state temperatures is independently maintained for a respective measurement period, measuring a first temperature at a first surface on the first side, and heat flow through the material, during each of the respective measurement periods, calculating R-values of the material at each of the three or more steady-state temperatures, and determining the R-value of the material at the mean temperature from the calculated R-values.

According to one aspect of this disclosure, there is provided a system for non-destructive in-situ determination of the R-value of a material at a mean temperature, the system including a housing defining a chamber and including an open end for facing the material, a temperature control system for heating and cooling inside the chamber to adjust between three or more steady-state temperatures in the chamber, one or more heat flux transducers for measuring heat transfer across the material within the chamber, one or more temperature sensors for measuring a temperature within the chamber, and a computing device for controlling the temperature control system, and the computing device including a data acquisition system for recording measurements from the heat flux transducers and the temperature sensors.

Other aspects and features of the present disclosure will become apparent to those of ordinary skill in the art upon review of the following description of specific embodiments.

### BRIEF DESCRIPTION OF THE DRAWINGS

For a more complete understanding of the disclosure, reference is made to the following description and accompanying drawings, each of which are intended to be non-limiting, in which:

FIG. 1 is a flowchart illustrating the steps of an embodiment of a method for non-destructive in-situ determination of the R-value of a material at a mean temperature;

FIG. 2 is a schematic of an embodiment of a system for non-destructive in-situ determination of the R-value of a material at a mean temperature;

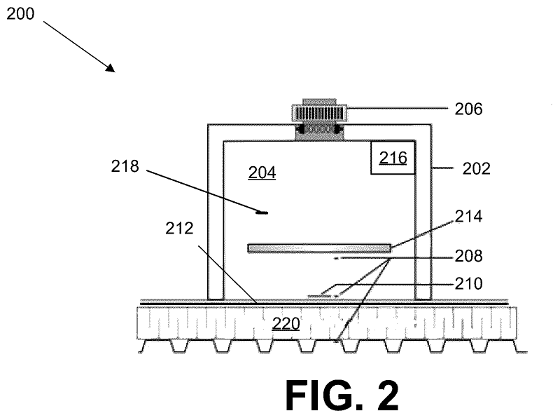

FIG. 3 is a graph illustrating air and surface temperature during a typical test involving the system of FIG. 2;

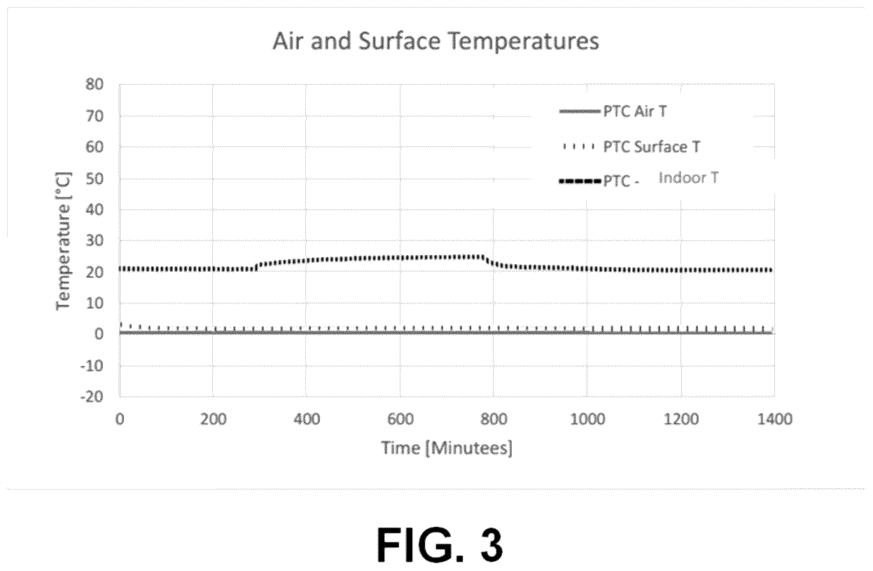

FIG. 4 is a graph illustrating heat flux through a material during a typical test involving the system of FIG. 2;

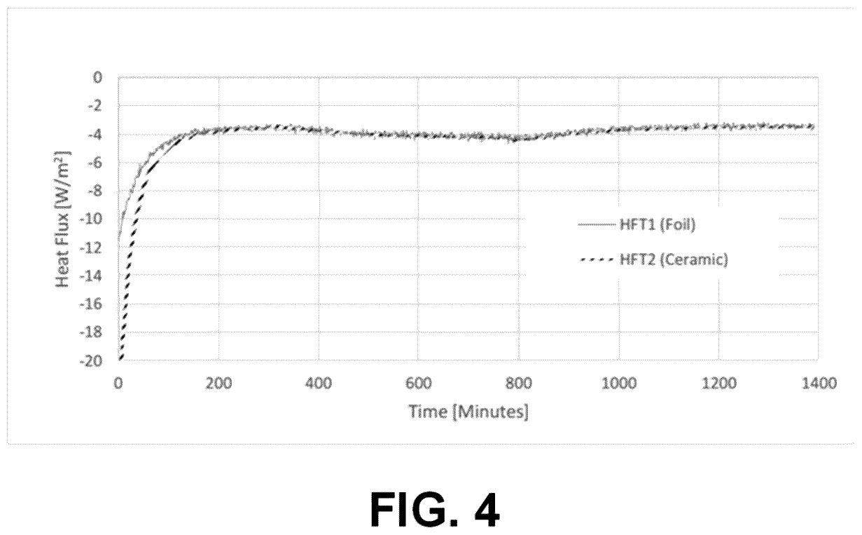

FIG. 5 is a graph illustrating calculated thermal resistance of a material during a typical test involving the system of FIG. 2;

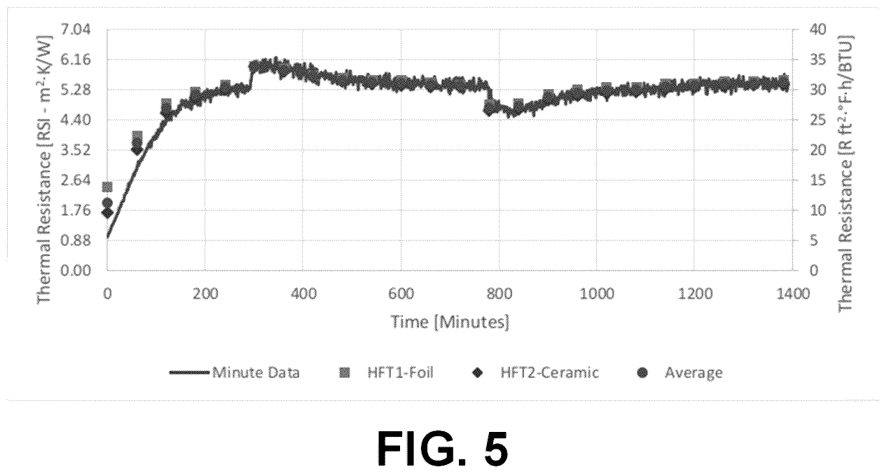

FIG. 6A is a graph illustrating the heat flux transducer calibration for a Hukseflux™ HFP01 heat flux transducer; and

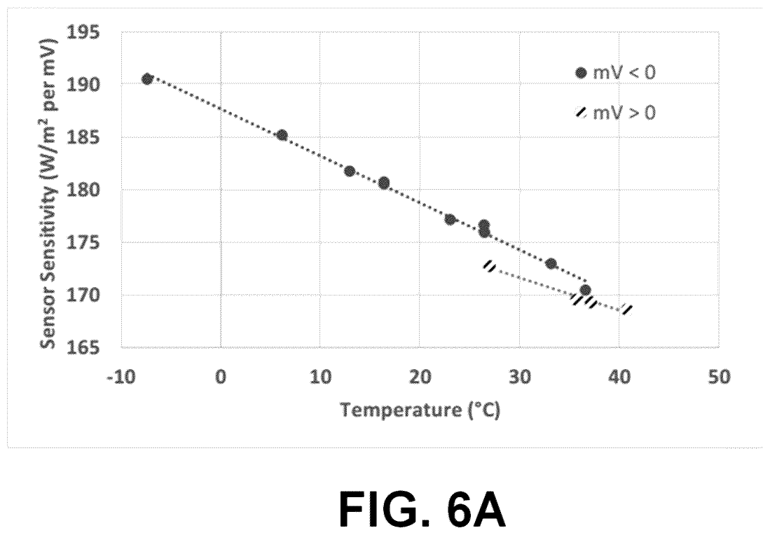

FIG. 6B is a graph illustrating the heat flux transducer calibration for a Hukseflux™ FHF02 heat flux transducer.

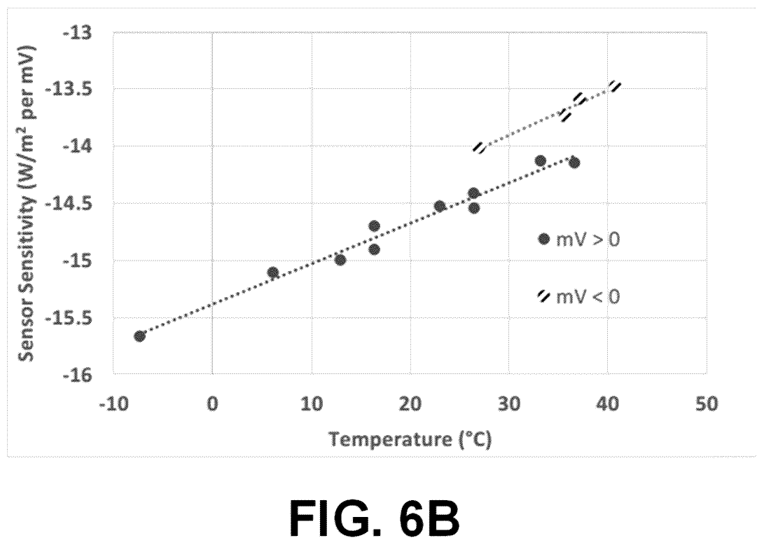

### DETAILED DESCRIPTION

Unless otherwise defined, all technical and scientific terms used herein generally have the same meaning as commonly understood by one of ordinary skill in the art to which this disclosure pertains. Exemplary terms are defined below for ease in understanding the subject matter of the present disclosure.

### Definitions

The term “a” or “an” refers to one or more of that entity; for example, “a temperature sensor” refers to one or more temperatures sensors or at least one temperature sensor. As such, the terms “a” (or “an”), “one or more” and “at least one” are used interchangeably herein. In addition, reference to an element or feature by the indefinite article “a” or “an” does not exclude the possibility that more than one of the elements or features are present, unless the context clearly requires that there is one and only one of the elements. Furthermore, reference to a feature in the plurality (e.g. systems), unless clearly intended, does not mean that the systems or methods disclosed herein must comprise a plurality.

“About”, when referring to a measurable value such as an angle, a dimension, and the like, is meant to encompass variations of ±10%, ±5%, ±1%, ±0.5% or ±0.1% of the specified amount. When the value is a whole number, the term about is meant to encompass decimal values, as well the degree of variation just described. It is to be understood that such a variation is always included in any given value provided herein, whether or not it is specifically referred to.

“And/or” refers to and encompasses any and all possible combinations of one or more of the associated listed items (e.g. one or the other, or both), as well as the lack of combinations when interrupted in the alternative (or).

“Comprise” as is used in this description and in the claims, and its conjugations, is used in its non-limiting sense to mean that items following the word are included, but items not specifically mentioned are not excluded.

### Methods of Non-Destructive In-Situ Measurements

Embodiments herein disclose systems and methods for non-destructive in-situ determination of a roof assembly's design-U value or insulation R-values for energy certification, energy audits, and design for roof replacement or roof recovering. In some embodiments disclosed herein, the system is portable and provides a reliable assessment of in-situ measurements by decreasing uncertainty in boundary conditions both from heat gains and temperature fluctuations, making it a cost-effective and time efficient alternative to existing systems and methods. In some embodiments disclosed herein, systems and methods determine a R-value of a roof or roofing material in place on a building, whereby optionally the building has an internal temperature of between about 20 and about 25 degrees Celsius. In other embodiments disclosed herein, systems and methods determine a R-value of materials (e.g. insulation boards) delivered to the construction site allowing the contractors to conduct quality assurance of the insulation, and confirm the characteristics of what they have ordered and received on-site.

In an embodiment, the present disclosure relates to a method for non-destructive in-situ determination of the R-value of a material at a mean temperature, the method comprising the steps of maintaining three or more steady-state temperatures at a first side of the material, wherein each of the three or more steady-state temperatures is independently maintained for a respective measurement period, measuring a first temperature at a first surface on the first side, and heat flow through the material, during each of the respective measurement periods, calculating R-values of the material at each of the three or more steady-state temperatures, and determining the R-value of the material at the mean temperature from the calculated R-values.

As used herein, the term “non-destructive determination” refers to determination without a requirement of removing, separating, deconstructing, disassembling or otherwise taking apart the material from other materials or structures that it is otherwise attached to, whether permanently attached or not. The term “in-situ” refers to the ability to perform a determination on a material, such as a roofing material, without moving it, for example, to a testing apparatus or testing facility. In an embodiment, by “in-situ” it is meant on-site where the material is intended to be used in construction and/or where the material is already in place in a construction (e.g. a building).

As used herein, the “mean temperature” may be any desired mean temperature at which the R-value of the material may be determined. In relation to building construction and materials used for such purposes, the typical requirement is that the mean temperature must be 75 degrees Fahrenheit (24 degrees Celsius). Thus, in some embodiments, the mean temperature is 24 degrees Celsius. However, other mean temperatures may be desired in certain circumstances. Thus, in some embodiments, the mean temperature may for example be any temperature between 0 and 50 degrees Celsius.

As used herein, the term “material” is intended to encompass any composition of matter for which it is desired to determine the R-value therethrough. The term “material” may refer to a single material, or collectively to a structure comprised of multiple materials, for example a layered or formed assembly (e.g. a roof). In an embodiment, the material may be a building material, an insulation material, a wood material, a glass material, a metal material, a composite material, a polymer material, or any other solid material. In some embodiments, the material is a roof, wall or window of a building, or a material that is used to construct these structures. In some embodiments, the material is a roof or roofing material. The roof or roofing material may for example comprise or consist of spray-on roofing (SPF), rolled roofing, built-up roofing, membrane roofing, asphalt composite shingles, metal roofing (e.g. metal tile sheets, standing seam metal roofing, metal shingles or shakes, etc.), wood shingle or shakes, clay tiles, concrete tile, slate shingles, or synthetic (e.g. rubber) slate tile. Metal roofing may, for example, be made from corrugated galvanized steel, aggregates of zinc, aluminum, or silicon-coated steel. By reference herein to “a roof”, it is intended to encompass multiple layers of roofing materials that form the roof from exterior to interior spaces, as opposed to a single roofing material.

In some embodiments, one or more locations are selected for thermal investigation based on building requirements and agreements between interested parties. Locations can be selected at a corner zone or perimeter zone away from a parapet wall, and to minimize thermal bridging at the selected location. Areas surrounding a roof drain may be avoided as the presence of tapered insulation could introduce errors in thermal resistance measurements.

The methods herein involve a step of maintaining three or more steady-state temperatures at a first side of the material at one or more locations. In some embodiments disclosed herein, the step of maintaining the three or more steady-state temperatures at the first side of the material is performed using the system as disclosed herein. Once the locations are selected, temperature sensors and heat flux transducers can be installed on the first side of the material. The heat flux transducers can be installed to the first side of the material using contact tape. In some embodiments disclosed herein, the temperature sensors and the heat flux transducers are separated by a distance, for example by at least six inches. In some embodiments, a membrane may then be placed over the heat flux transducers to cover them. In some embodiments, a humidity sensor is installed within the chamber. The temperature sensors, the heat flux transducers and the humidity sensors are connected to a computing device and/or a data acquisition system. The system includes a housing defining a chamber and including an open end for facing the material. Within the chamber, the temperature can be maintained at a steady-state over a given period of time (e.g. over the measurement period), and can be adjusted to different steady-state temperatures for purposes of performing the methods herein. The chamber may be of any suitable size, with exemplary non-limiting sizes being disclosed herein.

In some embodiments, the housing is then assembled, if applicable, and an enclosed chamber comprising an open end is placed over the temperature sensors, the heat flux transducers and the humidity sensors, with the heat flux transducers preferably being located close to the center of the chamber, with the open end against the first side of the material. In some embodiments disclosed herein, the housing comprises a plurality of modular panels connected using clip and lock mechanisms. The housing can be sealed along its housing against the roof system or roofing material to control heat loss and/or air/water entry. As the system may be obtaining measurements for a lengthy period of time (for example, five days), in some embodiments disclosed herein, the system is covered with a cover to provide protection from environmental conditions and to assist in maintain the steady-state temperatures. In some embodiments disclosed herein, the cover is weatherproof and/or waterproof. In some embodiments disclosed herein, the cover is supported by weighted elements to reduce movement and/or tipping in the event of wind or other environmental conditions. In some embodiments, a weighted element rests atop and/or is attached to the cover. In some embodiments disclosed herein, the cover comprises one or more vents to prevent any heat build-up inside and for circulation of air. In some embodiments disclosed herein, the housing is weatherproof or waterproof and/or the computing device including the data acquisition system are placed in a weatherproof or waterproof enclosure.

In some embodiments, the methods herein further comprise a step of measuring a second temperature during each of the respective measurement periods at a second side of the material, the second side being on an opposite side of the material than the first side. In some embodiments, the second temperature may be measured at or in close proximity to a second surface of the material, the second surface being on an opposite side of the material than the first surface. In other embodiments, such as when the material is already installed in a building, the second temperature may be measured anywhere within the building, preferably still in close proximity to the second surface. In a particular embodiment, such as when the material is a roof or roofing material, the second temperature may be measured at a steel deck under the roof or roofing material. One or more temperature sensors for measuring temperature can be installed using contact tape. In some embodiments, an indoor temperature monitoring logger can be used to record indoor temperatures. In some embodiments, the temperature sensors are connected to the computing device and/or the data acquisition system.

In some embodiments, the second temperature is between 10 and 30 degrees Celsius, more particularly between 20 and 25 degrees Celsius. In some embodiments, the second temperature is about 15, about 16, about 17, about 18, about 19, about 20, about 21, about 22, about 23, about 24, about 25, about 26, about 27, about 28, about 29 or about 30 degrees Celsius. In a particular embodiment, the second temperature is about 21 degrees Celsius. In a particular embodiment, the second temperature is about 22 degrees Celsius. In a particular embodiment, the second temperature is about 23 degrees Celsius. In a particular embodiment, the second temperature is between 20 and 25 degrees Celsius when the determination of the R-value is at a mean temperature of 24 degrees Celsius.

In some embodiments, the first surface is an exterior surface and the second surface is an interior surface. By “exterior surface” it is meant the side of the material that is intended to be or is exterior-facing in respect of a building or other construction. In contrast, by “interior surface” it is meant the side of the material that is intended or is interior-facing in respect of a building or other construction. For example, in an embodiment, the first surface is an exterior surface of a roof or a roofing material and the second surface is an interior surface of a roof or a roofing material.

The methods disclosed herein involve maintaining three or more steady-state temperatures independently, each for a respective measurement period. The measurement period may be any suitable time to accurately measure temperatures and heat flow through the material. The measurement period should be at least a sufficient period of time to obtain the steady-state temperature on the first side of the material. In an embodiment, the measurement period is between about 24 hours and about 72 hours. In some embodiments, each respective measurement period is independently a time of between about 24 hours and about 48 hours. In some embodiments, each of the respective measurement periods is about 24 hours.

In some embodiments, each of the measurement periods begins with a temperature ramp wherein the temperature control system works towards a steady-state temperature. During each measurement period, there is an initial period of fluctuation, which may be between 8 to 12 hours, wherein the temperature within the chamber and the roof are reaching equilibrium. This duration may be longer in roofs having a higher thermal mass, such as high-density mineral wool insulation or concrete decks. Duration of fluctuation depends on various factors, including the presence of moisture, contact with the membrane and roof components. The system is considered to be within a stabilization state once change in temperature differential and thermal resistance is less than about 10%.

As for the steady-state temperatures, these temperatures may be any suitable temperature and are typically determined based on the desired mean temperature at which the R-value is to be determined. For example, in some embodiments herein, the steady-state temperatures are differing values above and below the desired mean temperature. In an embodiment, at least one of the steady-state temperatures is below the desired mean temperature and at least one of the steady-state temperatures is above the desired mean temperature. In some embodiments, the methods disclosed herein involve multiple steady-state temperatures both below and above the desired mean temperature.

In some embodiments herein, at least one of the three or more steady state temperatures is below ambient. In some embodiments herein, at least one of the three or more steady state temperatures is below zero degrees Celsius. In some embodiments, each of the three or more steady-state temperatures are between −20 and 70 degrees Celsius, and more particularly between −10 and 60 degrees Celsius. In an embodiment, each of the three or more steady-state temperatures are a temperature of about −10, about −5, about 0, about 5, about 10, about 15, about 20, about 25, about 30, about 35, about 40, about 45, about 50, about 55, about 60, about 65, or about 70 degrees Celsius.

In some embodiments, the number of steady-state temperatures and the number respective measurement periods is at least three. In an embodiment, the number of steady-state temperatures and the number respective measurement periods is 3, 4, 5, 6, 7, 8, 9, 10, or more. In some embodiments, the number of steady-state temperatures and the number respective measurement periods each comprise three. In some embodiments, the number of steady-state temperatures and the number respective measurement periods each comprise five. As described herein, the number of measurement periods required in an instance may depend on characteristics of the material being evaluated.

In some embodiments, a first steady-state temperature is between −15 and −5 degrees Celsius, a second steady-state temperatures is between −5 and 0 degrees Celsius, a third steady-state temperatures is between 30 and 45 degrees Celsius, a fourth steady-state temperatures is between 45 and 60 degrees Celsius, and a fifth steady-state temperatures is between 60 and 70 degrees Celsius.

The methods herein involve a step of calculating R-values of the material at each of the three or more steady-state temperatures. Methods and systems disclosed herein use heat transfer calculations to provide non-destructive in-situ R-value measurements. The working principle of the system is to create a temperature differential (ΔT) across the material or roof assembly in an enclosed chamber, and measure the heat flow (Q) through the material or roof assembly. With these two parameters known, the thermal resistance of the roof assembly (R) can be determined using the following relationship:

$$R = Δ T / Q \qquad ( 1 )$$

By summing the thermal resistance of both indoor and outdoor air films to the measured thermal resistance value, an effective R-value of a roof assembly can be determined.

In some embodiments disclosed, steady-state thermal resistance at each mean temperature is determined from heat flow and temperatures recorded over a period of approximately 24 hours. Procedures of ISO 9869 (ISO, 2014) can be followed to determine thermal resistance using an average method. The average method determines thermal resistance by dividing the sum of the surface temperature differences by the sum of the heat flux through the material, as follows:

$$R = \frac{{\sum }_{j = 1}^{n} ( {T}_{s i j} - {T}_{s e j} )}{{\sum }_{j = 1}^{n} {q}_{j}} \qquad ( 2 )$$

The present disclosure provides systems and methods for calculating an R-value of a material at three or more steady-state temperatures to determine an R-value at a mean temperature, where directly calculating the R-value at the mean temperature is impracticable. For example, in commercial roofing, insulation is the primary thermal barrier of a roof. An insulation's R-value is generally measured in a laboratory at a mean temperature of 24° C. (average of the hot and cold temperatures, for example, 35° C. and 13° C.) with a temperature gradient of at least 20° C. across the insulation. The measured R-value at a mean temperature of 24° C. is commonly the labelled or reported R-value by manufacturers (and is a Federal Trade Commission rule) used in the thermal design of roofs to demonstrate compliance with energy codes. Measuring the in-situ R-value of the roof at this reporting mean temperature 24° C. and with the required temperature gradient of 20° C. is difficult since the building's indoor temperature is always maintained at about 20±2° C. or 20±5° C. Another way to measure the R-value at this mean temperature is to measure the R-value of the roof at other different temperatures, and through the regression analysis of the measured data (trend line fit), estimate the R-value at the reporting temperature of 24° C. There already exists an established relationship between the insulation's thermal conductivity and the mean temperature—a polynomial profile in polyisocyanurate insulation and linear profile with all the other insulation materials. Therefore, estimating the R-value at the reference temperature of 24° C. is an approach for reporting the roof's in-situ R-value.

In some embodiments disclosed herein, measurements are obtained at three or more steady-state temperatures. The number of steady-state temperatures depends on a number of factors including whether the insulation in question has a polynomial profile in polyisocyanurate insulation and linear profile with all the other insulation materials. Insulation having a polynomial profile generally requires five steady-state temperatures and insulation having a linear profile generally requires three steady-state temperatures for accurate calculations. Where the insulation material is known, either a polynomial or linear profile can be assumed as a characteristic prior to taking measurements to select the number of steady-state temperatures required and prior to performing calculations to reduce computational load. In some embodiments disclosed herein, at least one of the steady-state temperatures is below ambient. In some embodiments disclosed herein, at least one of the steady-state temperatures is below 0° C. In an example embodiment, measurements are obtained at steady-state temperatures of −2° C., −12° C., 42° C., 54° C. and 64° C. In some embodiments disclosed herein, measurements are obtained at five steady-state temperatures, wherein a first steady-state temperature is between −15° C. and −5° C., a second steady-state temperatures is between −5° C. and 0° C., a third steady-state temperatures is between 30° C. and 45° C., a fourth steady-state temperatures is between 45° C. and 60° C., and a fifth steady-state temperatures is between 60° C. and 70° C.

In an embodiment disclosed herein, the test procedure described above was used for a series of five measurements to determine the thermal resistance of the roof assembly at five different mean test temperatures. These mean test temperatures can be used to determine the R-value of the roof assembly at a reference mean temperature of 24° C.

In an embodiment disclosed herein, the R-value of the roof assembly at a reference mean temperature of 24° C. is determined using a spreadsheet. The measured data and the indoor temperatures are input into a spreadsheet for analyzing the data including using built-in macros. In some embodiments disclosed herein, a stabilization range is identified over which change in temperature differential and thermal resistance is less than 10%. Once the stabilization range is identified, the mean temperature and thermal resistance values are calculated at their respective set-point temperatures. A summary of the relationship between thermal resistance and temperatures in a graphical form may be displayed. In some embodiments disclosed herein, the data is analyzed by following a least-squares fit as follows:

For linear relationships (e.g. EPS, XPS and stone wool):

$$R = a * T + b , \qquad ( 3 )$$

where T is mean test temperature, and
a and b are equation coefficients.

For polynomial relationships (e.g. polyiso and spray foam):

$$R = a * T \hat{} 3 + b * T \hat{} 2 + c * T + d , \qquad ( 4 )$$

where T is mean test temperature, and
a, b, c and d are equation coefficients.

From equations (3) and (4), the R-value of the roof assembly is estimated for a mean temperature of 24° C.

In some embodiments, the methods herein further comprises a step of calibrating measurement devices. For example, prior to operating the system, in some embodiments disclosed herein, the heat flux transducers, the temperature control system and the temperature sensors are calibrated to measure their sensitivity when connected to the data acquisition system as a function of temperature. In an embodiment, to conduct calibration, the heat flux transducers are installed on a extruded polystyrene (XPS) insulation and connected to a heat flow meter, such as a Texas Instruments™ FOX 600. In some embodiments disclosed herein, the heat flux transducers are calibrated over a series of temperatures. In some embodiments disclosed herein, the temperature control system and the temperature sensors are calibrated over a series of temperatures by confirming temperatures within the chamber with a test temperature sensor.

Temperatures (° C.)

| Upper Plate | Lower Plate | Mean | Delta |
|---|---|---|---|
| −10.0 | 10.0 | 0.0 | 20.0 |
| 0.0 | 40.0 | 20.0 | 40.0 |
| 10.0 | 30.0 | 20.0 | 20.0 |
| 15.0 | 25.0 | 20.0 | 10.0 |
| 10.0 | 50.0 | 30.0 | 40.0 |
| 20.0 | 40.0 | 30.0 | 20.0 |
| 25.0 | 35.0 | 30.0 | 10.0 |
| 20.0 | 60.0 | 40.0 | 40.0 |
| 30.0 | 50.0 | 40.0 | 20.0 |
| 35.0 | 45.0 | 40.0 | 10.0 |
| 30.0 | 10.0 | 20.0 | 20.0 |
| 40.0 | 10.0 | 25.0 | 30.0 |
| 40.0 | 20.0 | 30.0 | 20.0 |
| 45.0 | 15.0 | 30.0 | 30.0 |

In some embodiments disclosed herein, a least-squares approach is used to develop linear equations for each sensor for heat flow in positive and negative directions. As a result, the sensitivity is based on a linear equation of the following form:

$$S = a * T + b , \qquad ( 5 )$$

where S is sensitivity of transducer (W*m⁻²-mV⁻¹),
T=transducer temperature, and
a and b are equation coefficients (W*m⁻²-mV⁻¹*C⁻¹ and W*m⁻²-mV⁻¹).

The following table shows linear fit results for both sensors and the equation (5) was used for measuring the heat flow through the system.

| | Heat Flow | HFS Sensitivity Coefficients | |
|---|---|---|---|
| Sensor | Direction | a | b |
| HFP 01 Ceramic Sensor | Positive | 0.0354 | −15.38 |
| | Negative | 0.0392 | −15.08 |
| FHF 02 Foil Sensor | Positive | −0.308 | 180.9 |
| | Negative | −0.446 | 187.6 |

In an embodiment disclosed herein, data from the data acquisition system and the indoor temperature monitoring logger are downloaded to a computer. In some embodiments disclosed herein, the computing system comprises an input interface, an output interface and performs and displays R-value calculations.

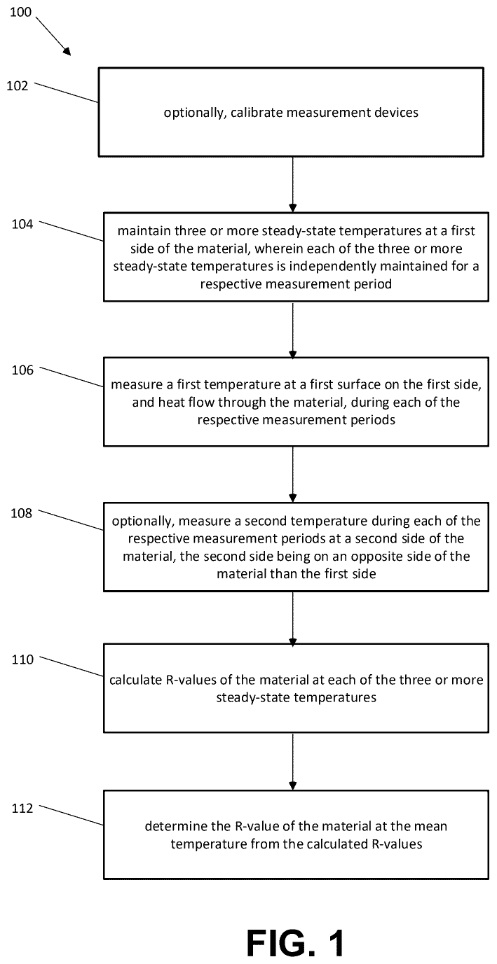

FIG. 1 illustrates the steps of an embodiment of a method 100 for non-destructive in-situ determination of the R-value of a material at a mean temperature. The method 100 optionally begins with calibrating measurement devices at step 102. At step 104, three or more steady-state temperatures are maintained at a first side of the material, wherein each of the three or more steady-state temperatures is independently maintained for a respective measurement period. At step 106, a first temperature at a first surface on the first side is measured, and heat flow through the material is measured, during each of the respective measurement periods. At step 108, optionally, a second temperature is measured during each of the respective measurement periods at a second side of the material, the second side being on an opposite side of the material than the first side. At step 110, R-values of the material are calculated at each of the three or more steady-state temperatures. At step 112, the R-value of the material at the mean temperature is determined from the calculated R-values.

### Systems for Non-Destructive In-Situ Measurements

In an embodiment, the present disclosure relates to a system for non-destructive in-situ determination of the R-value of a material at a mean temperature, the system comprising a housing defining a chamber and comprising an open end for facing the material, a temperature control system for heating and cooling inside the chamber to adjust between three or more steady-state temperatures in the chamber, one or more heat flux transducers for measuring heat transfer across the material within the chamber, one or more temperature sensors for measuring a temperature within the chamber, and a computing device. The computing device for controlling the temperature control system and comprising a data acquisition system for recording measurements from the heat flux transducers and the temperature sensors.

As described elsewhere herein, in some embodiments the material upon which the system may be used is a roof or roofing material.

Advantageously, embodiments disclosed herein are portable, meaning the system can be taken on-site to measure the R-value of a material and/or can be carried to and used on top (e.g. a roof) of an existing building. In some embodiments, the system is a portable thermal chamber (PTC).

In some embodiments, the housing is comprised of a rigid material and/or a rigid frame, wherein the rigid material may comprise metal, plastic, wood, polymer, and/or composite materials. In some embodiments disclosed herein, the housing defines a chamber, which may be insulated and which may be any size appropriate to enclose components of the system (e.g. 0.4 m×0.6 m (1.5 ft×2 ft) or 0.6 m×0.6 m (2 ft×2 ft)). In some embodiments disclosed herein, the walls of the housing are comprised of a thermally insulated material, wherein the thermally insulated material may comprise metal, foil, plastic, glass, wood, foam, polymer, composite and/or any other suitable material including any combination thereof. In some embodiments, the walls of the housing comprise an exterior aluminum C beam frame, vacuum insulated panels and foiled faced insulation. In some embodiments, the vacuum insulated panels are adhered to the aluminum C beam frame and the foil faced insulation adhered to the vacuum insulation panels. In some embodiments, an aluminum tape is used to seal along the perimeter of the foil faced insulation to a flange of the aluminum C beam frame. In some embodiments, the walls are about 75 mm (3 inches) thick. In some embodiments disclosed, the walls comprise panels forming the housing, which are modular and are detachably connected, for example using clip and lock mechanisms, which allows the panels to be disassembled during transport and storage for increased portability.

In some embodiments disclosed, a method of using the system comprises identifying a location for R-value measurement on a roof, and installing a heat flux transducer on the roof surface. In some embodiments, the system is equipped with a temperature control system capable of creating temperatures in the range of −20° C. to 70° C. With controlled local heating in the system, a high steady-state temperature differential is induced across the roof with minimal fluctuations, thereby increasing the measurement accuracy of the R-value of the roof. The high-temperature gradient shortens the measurement duration in determining the R-value of the roof, and it also allows a user to conduct in-situ measurements in any climatic conditions. In some embodiments disclosed herein, the housing and chamber are rectangular but the housing and chamber may also be any appropriate geometric shape.

In some embodiments, the system comprises a cover for placement over the housing. In some embodiments herein, the cover, the housing and/or an enclosure for the computing device, including the data acquisition system, are weatherproof and/or waterproof. The cover, housing and/or enclosure may be comprised, coated and/or treated with a material that is weatherproof and/or waterproof. The housing and/or enclosure may also be designed with a shape or structure that provides or enhances weather and/or water proofing. In some embodiments disclosed herein, the cover comprises one or more air vents to prevent heat build-up within the cover and to provide air circulation between the housing and the external environment. In some embodiments disclosed herein, the housing may comprise a baffle to control airflow within the chamber.

In some embodiments, the temperature control system is mounted to the housing. The temperature control system is for heating or cooling the chamber to adjust between three or more steady-state temperatures in the chamber. In some embodiments, the temperature control system is a thermoelectric unit or cooler. The thermoelectric unit may be a single unit capable of both heating and cooling or may be comprised of two or more units, each unit for heating or cooling.

In some embodiments, a temperature control system of the system can produce various temperature set points inside the chamber maintained at a steady state in the housing. In some embodiments disclosed herein, the temperature control system can heat the chamber to about 60° C. or 70° C. and cool the chamber to about −10° C. or −20° C. With a constant temperature gradient across the roof at each set point, the heat transfer rate through the roof is measured, and the R-value is determined at the different mean temperatures. This design feature of the system allows a designer to determine the energy performance of the roof at different operating temperatures of the roof, and can also provide a point of comparison with the energy codes. The temperature control system can be a standard component or can comprise a custom-developed controller and software.

In some embodiments, the system is capable of determining the R-value of the material at the mean temperature of 24 degrees Celsius when an indoor temperature of a building having the materials affixed thereto is between 20 and 25 degrees Celsius.

1. A method for non-destructive in-situ determination of the R-value of a material at a mean temperature, the method comprising the steps of:
maintaining three or more steady-state temperatures at a first side of the material, wherein each of the three or more steady-state temperatures is independently maintained for a respective measurement period;
measuring a first temperature at a first surface on the first side, and heat flow through the material, during each of the respective measurement periods;
calculating R-values of the material at each of the three or more steady-state temperatures; and
determining the R-value of the material at the mean temperature from the calculated R-values.

2. The method of claim 1, further comprising a step of measuring a second temperature during each of the respective measurement periods at a second side of the material, the second side being on an opposite side of the material than the first side.

3. The method of claim 2, wherein the second temperature is between 20 and 25 degrees Celsius.

4. The method of claim 2, wherein the first side is an exterior side and the second side is an interior side.

5. The method of claim 2, wherein the second temperature is measured at or in close proximity to a second surface of the material, the second surface being on an opposite side of the material than the first surface.

6. The method of claim 1, wherein the material is a roof or roofing material.

7. The method of claim 1, wherein each of the respective measurement periods is about 24 hours.

8. The method of claim 1, wherein at least one of the three or more steady state temperatures is below ambient.

9. The method of claim 1, wherein at least one of the three or more steady state temperatures is below zero degrees Celsius.

10. The method of claim 1, wherein each of the three or more steady-state temperatures are between −20 and 70 degrees Celsius.

11. The method of claim 1, wherein each of the three or more steady-state temperatures are between −10 and 60 degrees Celsius.

12. The method of claim 1, wherein the number of steady-state temperatures and the number respective measurement periods each comprise three.

13. The method of claim 1, wherein the number of steady-state temperatures and the number respective measurement periods each comprise five.

14. The method of claim 13, wherein
a first steady-state temperature is between −15 and −5 degrees Celsius,
a second steady-state temperatures is between −5 and 0 degrees Celsius,
a third steady-state temperatures is between 30 and 45 degrees Celsius,
a fourth steady-state temperatures is between 45 and 60 degrees Celsius, and
a fifth steady-state temperatures is between 60 and 70 degrees Celsius.

15. The method of claim 1, wherein the step of maintaining three or more steady-state temperatures and the step of measuring the first temperature are performed from within an enclosed chamber having an open end positioned against a roof.

16. The method of claim 15 further comprising a step of placing a cover over the enclosed chamber to protect the chamber from exterior elements.

17. The method of claim 1 further comprising a step of calibrating measurement devices.

18. The method of claim 1, wherein the determining step comprises one or more steps of interpolating the R-value for the mean temperature using the R-values at each of the three or more steady-state temperatures.

19. The method of claim 18, wherein interpolating the R-value for the mean temperature uses known characteristics of an insulation material.

20. The method of claim 19, wherein based on the known characteristics either a linear profile or a polynomial profile is employed in interpolating the R-value.

21. The method of claim 1, wherein the mean temperature is 24 degrees Celsius.

22.-45. (canceled)
